(19) United States
(12) Patent Application Publication
(10) Pub. No.: US 2004/0068235 A1
(43) Pub. Date: Apr. 8, 2004

(54) Packaging system for a medical device and particularly a punctum plug insertion device

(76) Inventor: Clive T. Hallam, Memphis, TN (US)

Correspondence Address: Gordon & Jacobson, P.C., 65 Woods End Road, Stamford, CT, 06905, US

(21) Appl. No.: 10/265,589

(22) Filed: Oct. 7, 2002

(51) Int. Cl. A61M 29/00

(52) U.S. Cl. 604/191; 606/191

(57) ABSTRACT

A packaging system for an ophthalmic medical instrument, particularly a punctum plug insertion device having a punctum plug loaded thereon, is provided. The system includes a clear plastic tube containing the device and a sterile barrier end cap enclosing an open end of the tube. The end cap may be provided with visual indicia to indicate a size of punctum plug loaded on the insertion device. In addition, the instrument may be color coded to indicate a size of the plug loaded on the insertion device. A carton is also provided for storing the tubes.

### BACKGROUND OF THE INVENTION

1. Field of the Invention

This invention relates broadly to ophthalmic devices and packaging devices therefor. More particularly, this invention relates to an instrument for dilating a punctum and inserting a punctum plug therein and a sterile packaging system containing the instrument and which identifies a size of the plug.

2. State of the Art

The surface of the eye and the inner surface of the eyelid are moisturized by tears constantly produced by glands around the eye. A tiny hole, known as the lacrimal punctum, at the inner corner of each upper and lower lid margin drains the tears away through ducts for proper circulation.

Patients, including contact lens wearers, who suffer from dry eye, or insufficient tear production, experience a great deal of discomfort because of insufficient lubrication between the lens and the surface of the eye. One solution is to occlude or block the lacrimal duct to prevent tear drainage. Permanent lacrimal occlusion can be performed surgically, typically by closing the punctal opening, whereas reversible occlusion can be performed by inserting a tiny plug into a portion of the lacrimal duct, such as the punctum. A typical punctum plug includes a cylindrical or frustoconical body, and a coaxial bore at which it is coupled to the tip of an insertion instrument, and a head portion.

A variety of instruments are available for inserting lacrimal occluders such as punctum plugs and canalicular implants. For example, Lacrimedics, Inc. of Rialto, Calif., has sold a canalicular implant preloaded on the tip of a wire stuck into a piece of foam. The wire is used to push the implant into the canaliculus. After the implant is deeply seated, the wire is withdrawn. Such a simple device lacks the capability of dilating the punctum prior to implant or plug insertion. Dilation is preferred to prevent irritation and damage to the punctal tissue.

U.S. Pat. No. 5,741,292 to Mendius (assigned to Eagle Vision) describes a more complex device for punctal dilation and insertion. The instrument that includes a cylindrical body having a plug inserting tip at one end and an opposite punctum dilating end. An elongated button is arranged longitudinally along the body, and includes a slidable end and a fixed end positioned between the slidable end and the plug inserting tip. The button has an outwardly bowing inner surface spaced from the body such that when the button is pressed toward the body, the slidable end slides away from the plug inserting tip. A wire is fixedly attached to the slidable end of the button, and slidably extends within the body and protrudes out of the plug inserting tip. A conventional punctum plug is attached to the protruding end of the wire.

In operation, one hand of the physician is placed against the face of a plug recipient, and the lid of the eye receiving the plug is pulled down to expose the punctal opening. The instrument is held in the other hand and the dilating tip is used to dilate the punctum. The instrument must then be turned around so that the plug inserting tip is now facing the eye, and the plug can then be inserted into the dilated punctum. It is noted that the device must be turned around carefully to prevent inadvertently dropping the instrument and to prevent unwanted contact with the plug (which may contaminate or dislodge the plug). Moreover, it must be turned around quickly, as the punctal opening begins to constrict within approximately five seconds after dilation. Once the plug is positioned in the punctum, the plug is released from the instrument by depressing the buttons, which retracts the wire and frees the plug.

FCI Ophthalmics of Marshfield Hills, Mass. sells a Ready-Set™ punctum plug system which also includes a plug inserter and punctal dilator instrument. The instrument is generally similar to the Mendius device, but includes two-sided trigger with handles located on diametrically opposite sides of the body. When the handles are pressed toward each other, the pin is retracted into the body, thereby dislodging the plug from the pin.

While the mendius and FCI Ophthalmic instruments are effective for punctum plug dilation and insertion, it is desirable to have a dilating/inserting instrument that can be operated to dilate the punctum and insert a punctum plug without turning the device around; i.e., for convenience, for plug integrity, and for time considerations. In fact, many physicians fail to perform punctal dilation before plug insertion due to the perceived awkwardness of using an instrument to dilate prior to inserting the plug. Moreover, it is desirable to have a dilating and inserting instrument that can be used to dilate and insert two punctum plugs without reloading the device or handling additional devices in order to facilitate and expedite the insertion of punctum plugs into multiple eyes benefiting from punctal occlusion.

### SUMMARY OF THE INVENTION

It is therefore an object of the invention is to provide a punctum dilating and punctum plug inserting instrument that has a dilator and an inserter on a common end of the instrument.

It is another object of the invention to provide a punctum dilating and punctum plug inserting instrument that has multiple dilators and can be preloaded with multiple plugs.

It is a further object of the invention to provide a punctum plug dilator and inserter that maintains multiple plugs on a single instrument and thusly permits multiple dilations and plug insertions in a patient without necessity of interruption while opening, handling, and/or replacement with a second instrument.

It is an additional object of the invention to provide a punctum dilating and plug inserting instrument that protectively covers the punctum plug until after punctal dilation.

It is also an object of the invention to provide a punctum plug inserting instrument that encourages conscious decision as to whether to dilate the punctum prior to plug insertion.

It is yet another object of the invention to provide a punctum dilating and plug inserting instrument that can be operated with one free hand and without necessitating movement of another hand that is positioned against a recipient's face to hold down the eyelid of the recipient for punctal dilation and insertion of a punctum plug.

It is yet a further object of the invention to provide a punctum dilating and plug inserting instrument that reliably and smoothly releases the plug without disturbing the plug's inserted position.

It is still a further object of the invention to provide a punctum plug insertion instrument provided with indicia that easily identifies the size of the plug mounted thereon.

It is still another object of the invention to provide a punctum dilating and plug inserting instrument that is very economical to manufacture, so that it is disposable after a single use.

In accord with these objects, which will be discussed in detail below, a first embodiment of a punctum dilating and plug inserting instrument includes a preferably cylindrically tubular body having a transverse slot. A wire movably extends within a first end of the body and protrudes therebeyond such that a punctum plug may be mounted thereon. A plug release means is coupled to or integrated with the body and the wire. According to an embodiment, the plug release means includes a first portion longitudinally fixed relative to the body, preferably by clipping onto the outside of the body, a second portion located within the body and coupled to the wire, and a resilient trigger. The trigger arches outside the body and passes into the transverse slot of the body to bridge the first and second portions. When the trigger is pressed, the second portion of the plug release means moves away from the first end of the body and causes retraction of the wire into the body, thereby releasing the plug mounted on the end of the wire. A guide is preferably coupled to the first end of the body and provides a small path for stable longitudinal movement of the wire.

According to a preferred aspect of the invention, a preferably optically clear cap is friction fit over the first end of the body and/or the guide. The cap tapers to form a tip that is sized to be inserted into the punctum to dilate the punctum. In addition, the cap provides a protective enclosure for a plug loaded on the wire until the cap is removed. Furthermore, the clear cap provides a means by which the punctum plug can be visually identified while being protectively covered.

The instrument of the invention may be operated by a physician as follows. The physician holds the instrument in a first hand, and places a second hand on a cheek of the plug recipient and pulls down the eyelid to expose the punctal opening. With the punctal opening exposed, the physician moves the tip of the cap into the punctal opening, and dilates the opening. Then, the instrument is removed from the punctal opening and maneuvered to place the cap in the crook of the second hand (between the thumb and forefinger), still located on the cheek. The cap is held in the crook of the second hand and the remainder of the instrument is pulled away to expose the punctum plug. The instrument is then again maneuvered to the punctal opening and the plug is inserted therein. Once inserted, the trigger is pressed to withdraw the wire and release the plug.

From the above, it is appreciated that a single instrument is provided which does not need to be turned around can be used to dilate the punctum and insert the punctum plug. As such, insertion can be rapid and prior to punctal relaxation. In addition, the instrument is always stably positioned within a hand of the physician. Furthermore, the cap can be removed without removing the physician's second hand from the face and lid of the recipient. Moreover, the plug is protected until the insertion step.

Referring to a preferred aspect of the invention and with respect to various embodiment, a wire, a wire guide, a plug release means, and a dilator cap can be provided at both ends of the body of the instrument. As such, a single instrument can be used to dilate puncta at both eyes, and insert plugs therein without necessitating an additional instrument or reloading of the instrument with an additional plug or even release of the instrument from a single hand of the physician.

Referring to yet other embodiments, the body may be solid and include a fixed pin at an end thereof. The plug release means includes a first portion longitudinally fixed to the body, a second portion which defines a sheath that may be moved over the fixed pin, and a trigger therebetween. A plug is provided on the fixed pin. When the trigger is actuated, the sheath is moved longitudinally and pushes the plug off the pin to thereby release the plug. A removable dilator cap fits over the sheath. This embodiment can likewise be provided in a two-ended version.

Additional objects and advantages of the invention will become apparent to those skilled in the art upon reference to the detailed description taken in conjunction with the provided figures.

### BRIEF DESCRIPTION OF THE DRAWINGS

FIG. 1 is a broken side elevation view of a punctum plug inserting instrument according to a first embodiment of the invention, shown with a trigger in a non-activated configuration and a punctum plug mounted at an end of the instrument;

FIG. 2 is an exploded view of a punctal dilator and plug inserting instrument according to the first embodiment of the invention, shown with a dilator cap;

FIG. 3 is a broken side elevation view of a punctum plug inserting instrument according to the first embodiment of the invention, shown with the trigger in an activated configuration and a punctum plug released therefrom;

FIG. 4 is an exploded view of a punctal dilator and plug inserting instrument according to a second embodiment of the invention, shown with dilator caps at each end;

FIG. 5 is a broken side elevation view of a punctum plug inserting instrument according to a third embodiment of the invention, shown with a trigger in a non-activated configuration and a punctum plug mounted at an end of the instrument;

FIG. 6 is an exploded view of a punctal dilator and plug inserting instrument according to the third embodiment of the invention, shown with a dilator cap;

FIG. 7 is a broken side elevation view of a punctum plug inserting instrument according to the third embodiment of the invention, shown with the trigger in an activated configuration and a punctum plug released therefrom; and

FIG. 8 is an exploded view of a punctal dilator and plug inserting instrument according to a fourth embodiment of the invention, shown with dilator caps at each end.

FIG. 9 is a side elevation view of a punctum plug inserting instrument according to a fifth embodiment of the invention, shown with a trigger in a non-activated configuration, a punctum plug mounted at an end of the instrument, a dilating cap in section shown at one end and a dilating cap shown in a removed position from the opposite end;

FIG. 10 is an enlarged side elevation of one end of the fifth embodiment of the instrument of the invention shown in the non-activated configuration;

FIG. 11 is an enlarged side elevation of one end of the fifth embodiment of the instrument of the invention shown in the activated configuration;

FIG. 12 is an individual packaging system for the instrument of the invention; and

FIG. 13 is a broken view of a packaging system for a plurality of the individual systems shown in FIG. 12.

### DETAILED DESCRIPTION OF THE PREFERRED EMBODIMENTS

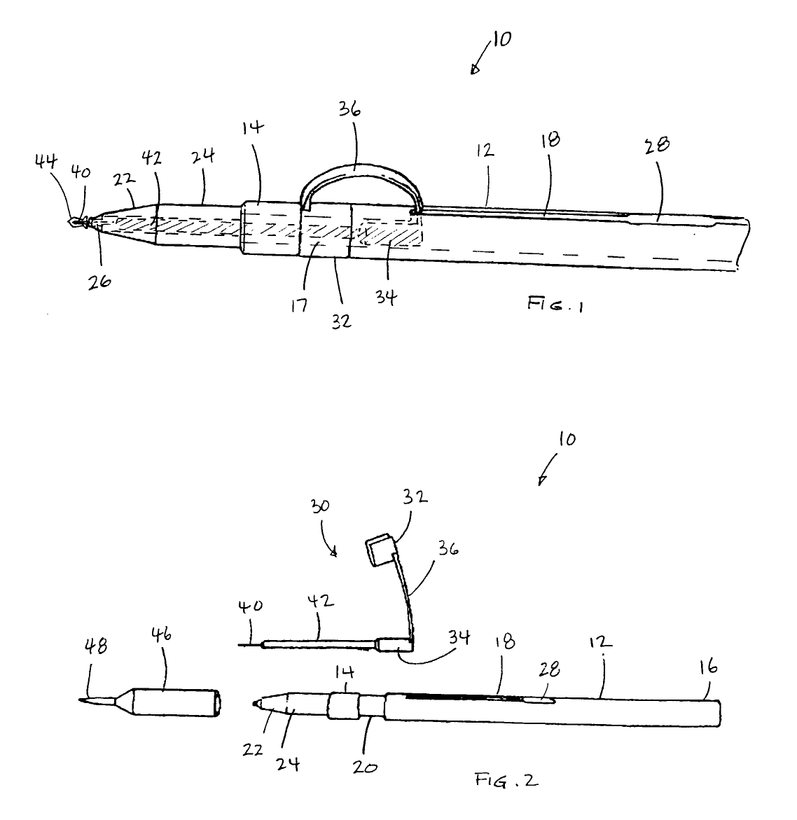

Turning now to FIGS. 1 and 2, a first embodiment of a punctum dilating and plug inserting instrument 10 includes a preferably plastic, cylindrical tubular body 12 having first and second end portions 14, 16, a longitudinal bore 17, and a transverse slot 18 entering into the bore 17. The first end portion 14 includes a preferably completely circumferential channel (i.e., a groove, slot or indent) 20; however, channel 20 may be only partially circumferential. The first end portion 14 also preferably tapers in diameter such that it terminates in a substantially frustoconical tip 22. The tapered tip 22 may be molded with the body 12, or may be provided as part of a distinct element 24 which is coupled to the body 12. The tip 22 includes a longitudinal bore 26 in communication with bore 17, but relatively smaller in diameter, and functions as a guide, as discussed below. An end of the transverse slot 18 located away from the first end portion 14 includes an enlarged opening 28.

The device 10 also includes a plug release element 30 having a clip portion 32, a wire mount 34, and a resilient trigger portion 36 therebetween. More particularly, the clip portion 32 is preferably a broken or slit cylindrical element adapted to be clipped about the channel 20 (i.e., on the outside of the body 12) to longitudinally fix the clip portion relative to the first end portion 14 of the body 12. The wire mount 34 is coupled to a preferably stainless steel wire 40 or other plug mountable element, e.g., a plastic pin, and is sized to be received through the enlarged opening 28 into the longitudinal bore 17, but not escape through the transverse slot 18. The wire 40 may be encased in a plastic sheathing 42 or otherwise supported along a substantial portion of its length. As shown in FIG. 1, the distal end of the wire 40 engages a punctum plug 44. The trigger portion 36 of the plug release element 30 arches outside the body 12 and passes into the transverse slot of the body to bridge the clip portion 32 and wire mount 34.

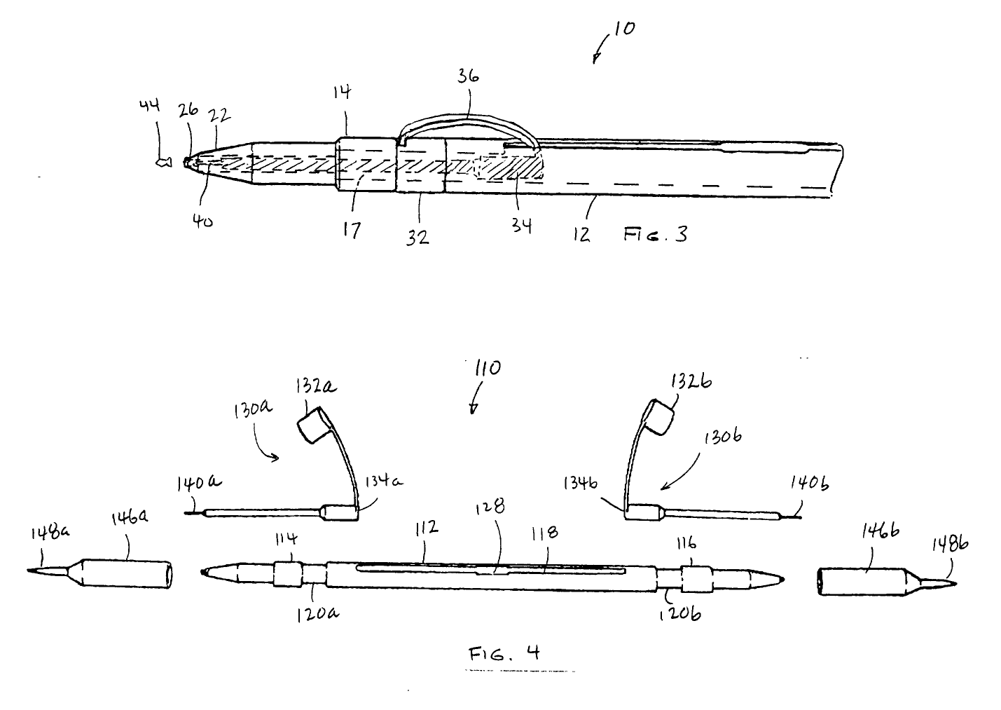

Referring to FIG. 3, when the trigger portion 36 is pressed toward the body 12, it elongates; i.e., it flattens, and the distance from one end to the other resultingly increases. As the clip portion 32 is longitudinally fixed in the channel 20 relative to the body 12, the wire mount 34 moves away from the first end 14 of the body as the trigger elongates. As a result, the wire 40 is retracted into the tip 22 of the body 12, and the plug 44 which is mounted on the end of the wire is too large to retract into the bore 26 of the tip, and is therefore released as the wire 40 is retracted. The bore 26 of the tip 22 defines a small path sized for stable longitudinal movement of the wire 40 when the trigger portion 36 is depressed; i.e., the tip 22 is a wire guide.

According to a preferred aspect of the invention, a cap 46 is removably friction fit over at least the tip 22 of the first end 14 of the body. The cap 46 tapers to form a dilator tip 48 sized to be inserted into the lacrimal puncta and dilate the sphincter muscles thereof. Alternatively, rather than taper, the cap 46 may be otherwise formed to have a dilator tip 48. The cap is preferably molded from a clear plastic material so that verification of the enclosed plug can be made. Thus, the cap 46 can be used to dilate the punctal opening, and is a protective enclosure for a plug loaded on the wire 40 until removed.

The described instrument of the invention may be operated by a physician as follows. The physician holds the instrument 10 in a first hand, and places a second hand on a cheek of the plug recipient and pulls away the eyelid to expose the punctal opening. With the punctal opening exposed, the physician, holding the instrument in the first hand, places the tip 48 of the cap 46 into the punctal opening to perform dilation thereof. Then, the instrument 10 is removed from the punctal opening and maneuvered to place the cap 46 in the crook (between the thumb and forefinger) of the second hand, still located on the cheek of the patient. The cap 46 is held in the crook and the remainder of the instrument is pulled away to expose the punctum plug 44. The instrument 10 is then again maneuvered to the punctal opening and the plug 44 is inserted therein. Once inserted, the trigger portion 36 is depressed to cause withdrawal of the wire 40 into the tip 22 of the instrument and release of the plug 44.

From the above, it is appreciated that a single instrument which does not need to be turned around can be used to dilate the punctum and insert a punctum plug. As such, insertion can be performed rapidly and before post-dilation punctal relaxation. In addition, the instrument is always stably positioned within a hand of the physician, as there is no need to turn the instrument around between the dilation and plug insertion procedures. Furthermore, the cap 46 can be removed without removing the physician's second hand from the face and lid of the recipient. Moreover, the plug 44 is protected by the cap 46 until, and verified within the cap prior to, the insertion step. In addition, the use of a clear dilator cap 46, showing the plug within, encourages conscious decision as to whether to dilate the punctum prior to plug insertion.

Turning now to FIG. 4, a second embodiment of a punctal dilator and punctum plug inserting instrument 110 according to the invention, substantially similar to the first embodiment 10, is shown. In accord with the third embodiment, the enlarged opening 128 of the transverse slot 118 is provided relatively centrally in the slot 118. Each of the first and second ends 114 and 116 of the body are provided with an at least partially circumferential channel 120a, 120b. Two plug release elements 130a, 130b are provided, and the clip portion 132a, 132b of each is coupled in the respective channel 120a, 120b. The wire mounts 134a, 134b can each be inserted into the body through enlarged opening 128. Each of the wire mounts 134a, 134b includes a wire 140a, 140b which extends out of a respective end portion 114, 116 of the body 112. A punctum plug (not shown) may be mounted on each wire 140a, 140b. Removable dilator caps 146a, 146b having respective punctum dilating tips 148a, 148b are provided over respective wires 140a, 140b and provide enclosures for the plugs mounted thereon. Each of the first and second end portions of the instrument functions in the same manner described above with respect to the first end portion of the first embodiment of the instrument. That is, with the instrument 110 of the second embodiment, a single instrument can be used to dilate the puncta at both eyes of a patient (or the upper and lower puncta of a single eye), and insert plugs therein without necessitating reloading of the instrument with an additional plug or even release of the instrument from a single hand of the physician.

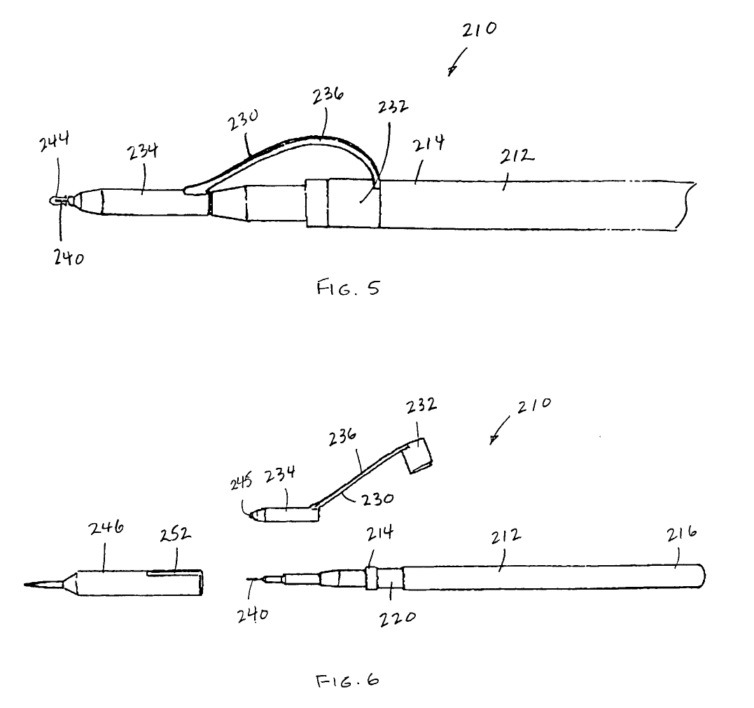

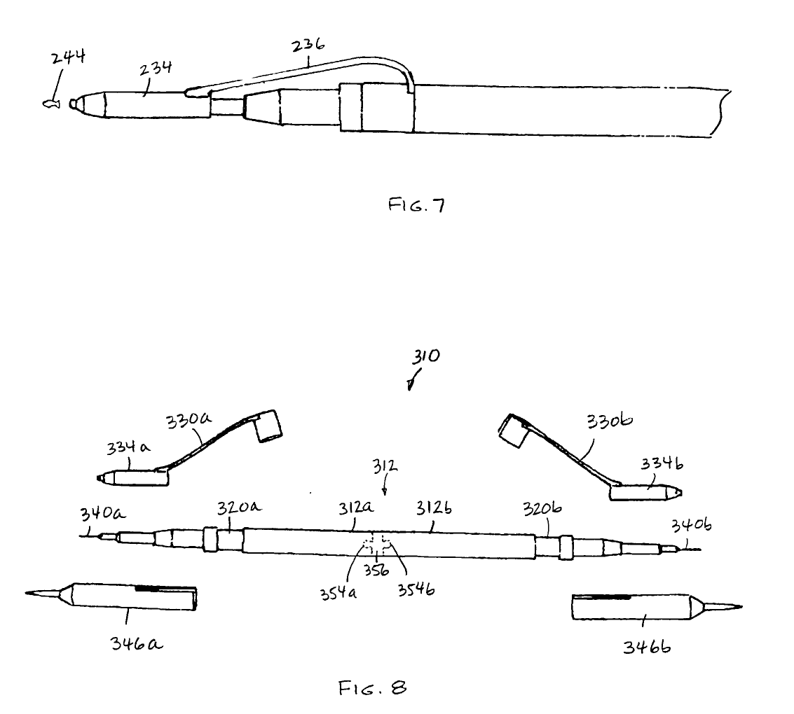

Referring now to FIGS. 5 and 6, a third embodiment of the instrument according to the invention is shown. The instrument 210 includes a preferably solid plastic body 212 having first and second ends 214, 216, and a wire 240 or a pin (not shown) fixed at the first end for mounting of a punctum plug 244 thereon. The wire 240 may be molded with the body 212 during manufacture, or a pin may be integrally formed of the same material as the body. The body 212 also includes a channel 220. A plug release element 230 includes a clip portion 232 clipped about the channel 220, a sheath 234, and a resilient trigger portion 236 therebetween. The sheath 234 is sized to move longitudinally over the first end of the body, including completely over the wire 240. Referring to FIG. 7, when the trigger portion 236 is depressed, the sheath 234 is moved away from the second end 216 of the body; i.e. in a release direction. An opening 245 at the end of the sheath 234 is sized such that when the sheath is moved over the wire 240 in the release direction, a punctum plug 244 positioned on the wire 240 is forced off the wire by the end of the sheath and released. A removable dilator cap 246 is preferably friction fit over the sheath 236. The cap 246 includes a slot 252 into which the trigger portion 236 may extend.

Turning now to FIG. 8, a fourth embodiment of the dilating and plug inserting instrument 310 according to the invention is shown. The fourth embodiment is a two-ended version in which each end is functionally similar to the first end of the third embodiment. As such, the body 312 includes two clip receiving channels 320a, 320b, and two wires 340a, 340b. Two plug releasing elements 330a, 330b are coupled to the body and includes sheaths 344a, 344b longitudinally movable relative to the wires 340a, 340b. In addition, two dilator caps 346a, 346b are provided, one for each end.

Furthermore, it is understood that any of the two-ended versions of the instruments may be comprised of two single-ended versions which are coupled back-to-back, in either a releasable coupling or permanent coupling. For example, and not by limitation, still referring to FIG. 8, the body 312 may comprise first and second distinct portions 312a, 312b each having a rear opening 354a, 354b. A coupling element 356 couples portions 312a, 312b in a preferably linear configuration. Alternatively, the first distinct portion 312a may include a rear male end, and the second distinct portion 312b may include a rear female end which can be coupled together without an intervening coupling element.

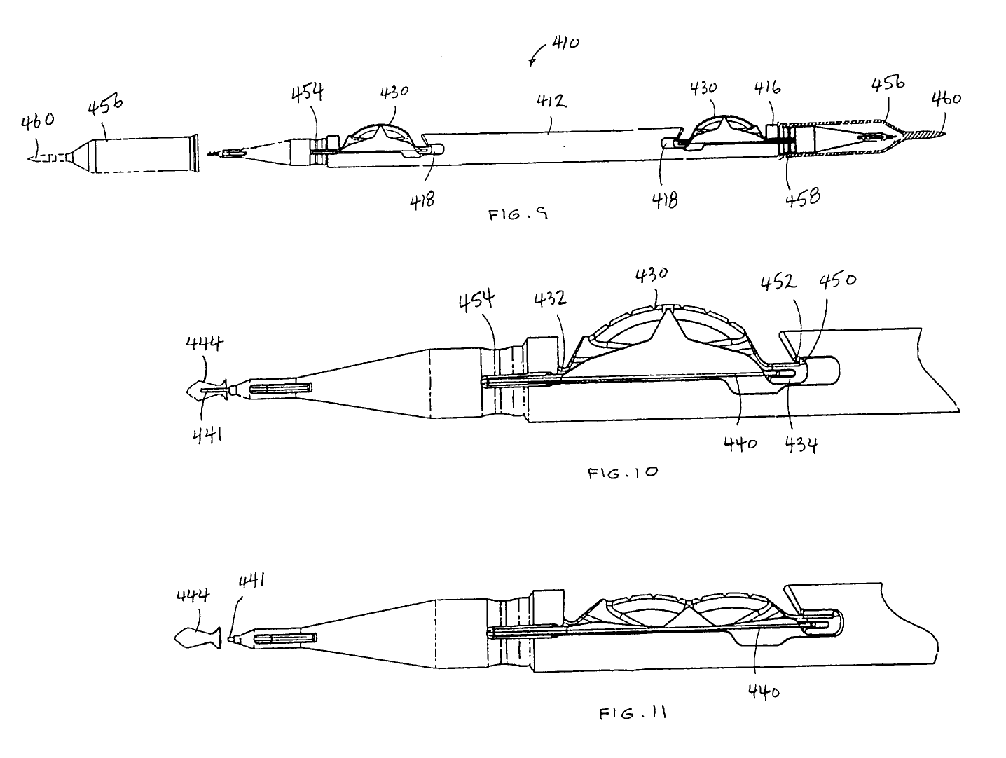

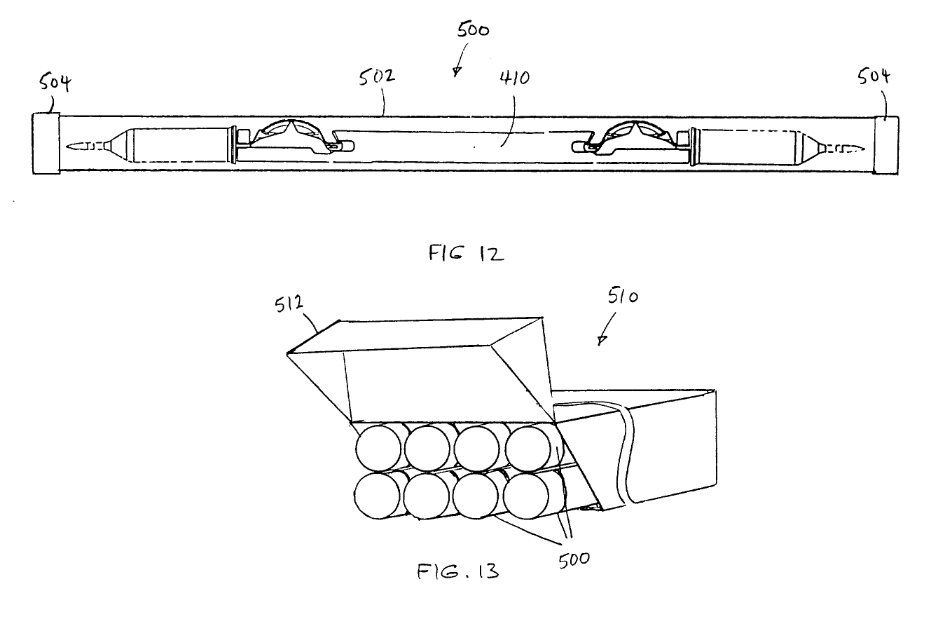

Referring now to FIGS. 9 and 10, a fifth embodiment of the instrument 410 is shown. The instrument 410 has a body 412 having two ends 414, 416 substantially as described above. Each end, e.g., end 414, preferably defines a compressible trigger 430 which arches radially outward from the body. One end 432 of the trigger is preferably integrally formed with the body 412, while a free end 434 of the trigger is movable within a slot 418. The free end 434 preferably includes a catch 450 which is inserted into the slot 418 and held captured therein by a barb 452 at the entrance to the slot. A wire 440 or pin is longitudinally fixed to the free end 434 of the trigger 430 and extends longitudinally through and out the end 414 of the body. A punctum plug 444 is mountable on the end 441 of the wire 440. Referring to FIG. 11, when the trigger 430 is depressed, the free end 434 of the trigger 430 is moved through the slot 418, causing the wire 440 to be at least partially withdrawn into the body 412 and thus release the punctum plug 444. Each end portion 414 of the body also includes a preferably circumferential groove 454. Referring back to FIG. 9, a removable cap 456 is provided for each end 414, 416 of the body. The caps 456 each include an inner preferably circumferential ridge 458 which frictionally engages in respective grooves 454 of the body. The caps each also define a dilating tip 460. The caps 456 can be used for plug verification and punctal dilation, and removed for plug access as discussed above with respect to the other embodiments.

Each of the instruments may be molded in a plastics of various colors or otherwise color coded, with each color associated with a single size of punctum plug which is mounted on the instrument. As such, a quick visual inspection of the instrument provides identification of the size of the plug thereon.

Turning now to FIG. 12, a packaging for the instrument of the invention is provided. The packaging 500 includes a preferably clear substantially rigid plastic tube 502 sized to accommodate the instrument (preferably with plug and cap) and two removable cap seals 504. By “substantially rigid”, it is meant that the tube is adapted to maintain its shape, but may have some resiliency. The cap seals 504 provide a sterile barrier and protection for the dilator tip of the instrument 410 and the plugs loaded thereon (not shown in FIG. 12). Referring to FIG. 13, a carton 510 is preferably provided that accommodates a plurality of thusly packaged instruments 500. The carton 510 preferably includes a flip open top 512 (similar to a box of crayons) or other removable top that permits easy access the packaged instruments. In addition, the carton is preferably constructed of cardboard, but may be made of a plastic or other suitable material. Instruments packaged in this manner (as opposed to the typical sterile pouch packaging), minimize shipping cost, conserve inventory space, and conveniently permit quick access to the instruments, as well as assessment of the number of instruments on hand. Moreover, the sterile packaged instrument requires less space on an instrument tray prior to use.

Because the tubes 502 are clear, the color-coded instrument contained within each can be seen therethrough. Additionally or alternatively, the cap seals 504 may be color coded to correspond to the instrument and/or identify the size of the punctum plug. Other visual indicia can also be used, such as black and white or colored patterns, stripes, dots, etc. Furthermore, the tubes 502 can be etched, e.g., with a laser, with catalog number (model number) and/or batch numbers for product identification and traceability. Moreover, it is appreciated that other ophthalmic devices, or even medical devices outside the ophthalmic field, can be packaged in a like manner.

There have been described and illustrated herein several embodiments of a punctal dilator and punctum plug inserting instrument, and a method of using the same to insert a punctum plug. While particular embodiments of the invention have been described, it is not intended that the invention be limited thereto, as it is intended that the invention be as broad in scope as the art will allow and that the specification be read likewise. Thus, while particular means for mounting the trigger to the body have been disclosed, it will be appreciated that other mounting means may be used as well, provided that the mounting means does not inhibit movement of the trigger and the wire. For example, means other than a channel on the body may be used to longitudinally fix a clip on the outside of the body. In addition, the trigger may be mounted to the body by inserting a portion of the plug release means into the body, by welding it thereto, etc. In addition, while a wire has been disclosed for mounting a plug at the end of the instrument, it will be appreciated that other pin-like mounting elements may be used in any of the embodiments. Also, while the cap is preferably friction fit on the end or ends of the instrument, it will appreciated that, while less desirable, the cap may be thread onto an end, snap fit over an end, or otherwise removably provided on an end. Furthermore, while various elements of the instrument are preferably made of plastic, for ease of manufacture and low cost, it will be appreciated that other materials, including metal, may be used. Moreover, it will be appreciated that various aspects of the several embodiments can be combined into yet other embodiments that all are within the scope of the invention. It will therefore be appreciated by those skilled in the art that yet other modifications could be made to the provided invention without deviating from its spirit and scope as claimed.

What is claimed is:

1. A packaged medical instrument, comprising:
a) a clear substantially rigid plastic tube having a first open end;
b) a first removable seal cap located at said first open end providing a sterile barrier at said first open end; and
c) an elongate medical instrument located within said tube.

2. A packaged medical instrument according to claim 1, wherein:
said medical instrument is an ophthalmic device.

3. A packaged medical instrument according to claim 1, wherein:
said medical instrument is a punctum plug insertion device.

4. A packaged medical instrument according to claim 3, wherein:
said punctum plug insertion device includes first and second ends, and a punctum plug is provided at each of said first and second ends.

5. A packaged medical instrument according to claim 1, wherein:
said tube has a second open end and a second removable seal cap is located at said second open end providing a sterile barrier at said second open end.

6. A packaged medical instrument according to claim 1, wherein:
said tube is etched with at least one of a model number and a batch number.

7. A packaged medical instrument according to claim 1, further comprising:
d) a second seal cap,
wherein said tube includes a second open end, and said second seal cap is located at said second open end and provides a sterile barrier at said second open end.

8. A set of packaged medical instruments, comprising:
a) a plurality of medical implant insertion devices;
b) a plurality of medical implants each having a one of a plurality of sizes, one of said medical implants loaded on each said insertion device;
c) a plurality of substantially rigid plastic tubes each having a first open end, and each containing one of said medical implant insertion devices; and
d) a first removable seal cap located at said first open end of each said tube providing a sterile barrier at said first open end, said, each said seal cap having visual indicia corresponding to a size of said medical implant on said insertion device located in said tube.

9. A set of packaged medical instruments according to claim 8, wherein:
said medical implants are ophthalmic implants.

10. A set of packaged medical instruments according to claim 8, wherein:
said medical implants are punctum plug insertion devices.

11. A set of packaged medical instruments according to claim 8, wherein:
said visual indicia is color.

12. A set of packaged medical instruments according to claim 8, further comprising:
e) a carton containing a plurality of said tubes, each provided with a punctum plug insertion instrument.

13. A set of packaged medical instruments according to claim 12, wherein:
said carton has a flip open top.

14. A set of packaged medical instruments, comprising:
a) a plurality of medical implant insertion devices;
b) a plurality of medical implants each having one of a plurality of sizes, one of said medical implants loaded on each said insertion device; and
c) a plurality of substantially clear and rigid plastic tubes each containing one of said medical implant insertion devices,
each said insertion device having visual indicia visible through said tube in which it is contained, said visual indicia indicating a size of said medical implant on said insertion device located in said tube.

15. A set of packaged medical instruments according to claim 14, wherein:
said plurality of medical implants are ophthalmic implants.

16. A set of packaged medical instruments according to claim 14, wherein:
said plurality of medical implants are punctum plug insertion devices.

17. A set of packaged medical instruments according to claim 14, wherein:
said visual indicia is color.

18. A set of packaged medical instruments according to claim 14, further comprising:
d) a carton containing said plurality of said tubes.

19. A set of packaged medical instruments according to claim 14, wherein:
said carton has a flip open top.
